(19) United States
(12) Patent Application Publication
(10) Pub. No.: US 2014/0011893 A1
(43) Pub. Date: Jan. 9, 2014

(54) COMPOSITION AND METHOD

(71) Applicant: Kjell Stenberg, Akers Styckebruk (SE)

(72) Inventor: Kjell Stenberg, Akers Styckebruk (SE)

(21) Appl. No.: 14/006,701

(22) PCT Filed: Mar. 21, 2012
(86) PCT No.: PCT/EP12/54986

§ 371 Date: Sep. 23, 2013

Related U.S. Application Data
(60) Provisional application No. 61/466,203, filed on Mar. 22, 2011.

(30) Foreign Application Priority Data
Mar. 22, 2011 (EP) 11159232.5

(51) Int. Cl. A61K 9/00; A61K 47/36; A61J 3/00

(52) U.S. Cl. CPC A61K 9/006; A61J 3/00; A61K 47/36
USPC 514/777; 427/2.31; 156/60

(57) ABSTRACT

A method of preparing a mucoadhesive film formulation by preparing a film forming composition including lambda carrageenan as a film forming polymer, at a concentration of about 1-7% by weight, a plasticizer at a concentration of about 1-15% by weight, a biologically active substance, and purified water; distributing the film forming composition as a wet film layer onto a solid surface; and allowing the film layer to dry. A mucoadhesive film obtained by the method and a film dosage unit.

### FIELD OF THE INVENTION

The present invention relates to a mucoadhesive film comprising at least one biologically active substance as well as to a method of preparing such a film. In particular, the invention relates to a mucoadhesive film comprising lambda carrageenan, a plasticizer and at least one biologically active substance for use in the transmucosal delivery of biologically active substances.

### BACKGROUND OF THE INVENTION

Mucoadhesive films as vehicles for delivery of biologically active substances to the body are known within the prior art. For example, US patent application No. 2009/0186107 describes an orally administrable mucoadhesive film which comprises one or more bioactive ingredients in combination with film-forming polymer. Said application makes a distinction between fast dissolving films (FD films) and films for use as mucosal delivery systems (MDS) and notes that while the former type eliminates the need of swallowing, use of water for administration, and hazard of choking, the latter type allows to accomplish first-pass administration (without passing the digestive tract) of biologically active substance to or via mucosal membranes. According to said application, FD films dissolve within seconds, while MDS films require a longer residence time of at least a few minutes in order to direct the biologically active substance flux towards the mucosal membrane and bloodstream.

Requirements of successful mucoadhsive delivery films generally are that:

they should accept large amounts of active agents per unit dose, either in solution or dispersed;

they should accept dispersed, insoluble fillers of different types, penetration enhancers such as detergents as well as various kinds of taste masking agents and colouring agents;

they should be easy to handle and to self-administer by users with minimal training;

they should be able to retain their tensile strength, softness, flexibility, be easy to handle and have a good mouth feel even at very high amounts of added substances;

they should be able to adhere firmly and rapidly to all types of wet mucosa such as the buccal, gingival and palate surface in the mouth as well to all other wet surfaces on or inside a body;

they should be slowly dissolving while remaining closely associated (in a viscous film) to wet mucosa to enable a high, local concentration of active agent(s) to be formed and maintained promoting an efficient transmucosal, systemic delivery;

they should dissolve completely and leave no uncomfortable residues at the site of delivery; they should not negatively affect the cell membranes where the film vehicle adheres and delivers agents;

they should allow delivery of drugs thorough mucosa with minimal discomfort (bad taste due to spread to the taste buds or, tissue irritation etc.) to the user

they should support the stability of active ingredients in the dry film to enable longer term storage of film products;

they should not need the addition of preservatives for microbial control; they should be manufactured using conventional manufacturing and packaging procedures at a low cost; and

they should be able to be folded, rolled, cut or otherwise processed into suitable shape.

It is an object of the present invention to provide a mucoadhesive film for mucosal delivery of a biologically active substance that essentially fulfils most of the above-mentioned requirements.

Carrageenans form a naturally-occurring family of high-molecular-weight polysaccharides extracted from red seaweed. They are made up of sulfated and nonsulfated repeating galactose and 3,6-anhydrogalactose (3,6-AG) units, joined by alternating alpha 1-3 and beta 1-4 glycosidic linkages. Carrageenans are widely used in the food and other industries as thickening and stabilizing agents. There are three basic types of carrageenans, viz. kappa, iota and lambda carrageenan. One of the main producers of carrageenans is FMC Biopolymer Ltd and characteristics of the three carrageenans, according to the producer, are the following:

Kappa Carrageenan:
Soluble in hot water
The addition of potassium ions induces the formation of a durable, brittle gel; it also increases the gelling and melting temperatures.
Strong, rigid gel, some syneresis, forms helix with K⁺ ions. Ca²⁺ causes helices to aggregate and the gel to contract and become brittle.
Slightly opaque gel. Becomes clear with sugar.
Approximately 25% ester sulfate and 34% 3,6-AG
Compatible with water miscible solvents
Insoluble in most organic solvents
Typical use levels—0.02 to 2.0%

Iota Carrageenan:
Dilute solutions exhibit thixotropic characteristics
Soluble in hot water; sodium iota carrageenan is soluble in cold and hot water
The addition of calcium ions will induce the formation of a durable, elastic gel, and increase gelling and melting temperatures.
Elastic gels, forms helix with Ca²⁺. Limited aggregation contributes to elasticity, no syneresis.
Clear gel
Freeze/thaw stable
Insoluble in most organic solvents
Approximately 32% ester sulfate and 30% 3,6-AG
Typical use levels range from 0.2 to 2.0%

Lambda Carrageenan:
Free flowing, non-gelling pseudo-plastic solutions in water
Partially soluble in cold water, fully soluble in hot water
No gel, random distribution of polymer chains
Range from low to high viscosity
Addition of cations has little effect on viscosity
Compatible with water miscible solvents
Insoluble in most organic solvents
Stable over a wide range of temperatures, including freeze/thaw cycles
Soluble in 5% salt solution, hot or cold
Approximately 35% ester sulfate and little or no 3,6-AG
Typical use level—0.1 to 1.0%

It appears that lambda carrageenan differs substantially from the two other carrageenans, both in its chemistry and by its physical characteristics. For example, whereas kappa and iota carrageenan both contain quite high amounts of 3,6-anhydrogalactose, lambda carrageenan contains virtually no 3,6-anhydrogalactose, and whereas kappa and iota carrageenan form gels, lambda carrageenan does not.

The use of lambda carrageenan in different pharmaceutical compositions has been disclosed in the prior art. For example, United States Patent Application No. 20080274191 discloses viscous liquid compositions for producing pasty forms having a prolonged action and/or release for local applications by the in-situ formation of a bioadhesive more or less viscous, biodegradable matrix film. The prolonged release is to take place over more than 2 hours, e.g. up to 12 hours. The composition contains at least one matrix agent, a medium for hydrating the matrix agent, and at least one active substance. Lambda-carrageenan is mentioned as an example of a matrix agent.

United States Patent Application No. 20070184093 describes soluble films composed of a combination of two polymers, i.e. a first, soluble polymer and a second, strengthening polymer. Lambda carrageenan is mentioned as one among a number of possible soluble polymers. The films are said to be useful e.g. as edible, orally dissolvable strips or in edible, orally dissolvable pouches, e.g. for pharmaceuticals.

### SUMMARY OF THE INVENTION

It is an object of the present invention to provide a mucoadhesive film for mucosal delivery of a biologically active substance that essentially fulfils most of the above-mentioned requirements for such films.

The present inventor has surprisingly found that lambda carrageenan may be used as sole film forming agent in the preparation of dry films that are e.g. easy to handle, have a good adherence to wet mucosa and have a suitable dissolution profile in contact with the wet mucosa. Moreover, the film of the present invention has several other advantageous features in line with the object of the invention, as will be apparent from the following detailed description.

Consequently, according to a first aspect, the present invention provides a method of preparing a mucoadhesive film by
preparing a film forming composition comprising

(i) lambda carrageenan at a concentration of about 1-7% by weight,

(ii) a plasticizer at a concentration of about 1-15% by weight,

(iii) a biologically active substance, and

(iv) purified water;
distributing the film forming composition of the invention as a wet film layer onto a solid surface; and
allowing the film layer to dry.

According to a further aspect, the present invention provides a mucoadhesive film formulation obtainable by the method of the invention.

According to a still further aspect, the present invention provides a dosage film unit comprising a piece of the inventive film formulation according to the invention.

The film of the invention is useful for oral, e.g. buccal, gingival or palatal, transmucosal administration of a biologically active ingredient, e.g. an ingredient having a therapeutic activity, and preferably dissolves within a time period of 1 minute to 15 minutes in contact with a mucosal surface.

It also is contemplated that the film may be used for transmucosal administration of a biologically active ingredient through any other accessible mucosal surface, such as the nasal, genital (vaginal, penile) or anal mucosa.

The present invention also relates to a dosage unit of the film according to the invention, e.g. in the form of a transmucosal patch.

Still further aspects of the invention and embodiments thereof will be apparent from the following detailed description including the Examples.

### BRIEF DESCRIPTION OF THE DRAWINGS

FIG. 1 is a schematic sectional view of a multilayer film preparation of the invention.

FIG. 2 is a schematic sectional view of another multilayer film preparation of the invention, taken along the broken line A-A of FIG. 6.

FIG. 3 is a schematic sectional view of another multilayer film preparation of the invention, taken along the broken line A-A of FIG. 7.

FIG. 4 is a schematic sectional view of multi-area monolayer film preparation of the invention.

FIG. 5 is a schematic sectional view of another multi-area monolayer film preparation of the invention.

FIG. 6 is a top plan view of a multilayer film preparation of the invention.

FIG. 7 is a top plan view of another multilayer film preparation of the invention.

As should be realized, the drawings are not drawn to scale and are only schematic.

### DETAILED DESCRIPTION OF THE INVENTION

According to a first aspect, the present invention provides a method of preparing a muco-adhesive film by
preparing a film forming composition comprising

(i) lambda carrageenan at a concentration of about 1-7% by weight,

(ii) a plasticizer at a concentration of about 1-15% by weight,

(iii) a biologically active substance, and

(iv) purified water;
distributing the film forming composition as a wet film layer onto a solid surface; and
allowing the film layer to dry.

In the film forming composition, lambda carrageenan is present as a film forming polymer. The film forming composition comprises lambda carrageenan at a concentration of at least 1% by weight, at least 1.5% by weight, at least 2% by weight, at least 2.5% by weight or at least 3% by weight; and at most 7% by weight, at most 6.5% by weight, at most 6% by weight, at most 5.5% or at most 5% by weight; based on the total weight of the film forming composition before drying. For example, the film forming composition may comprise 1 to 6.5% by weight of lambda carrageenan, or 1.5 to 6% by weight of lambda carrageenan, e.g. 2 to 5.5% by weight of lambda carrageenan, in particular 2.5 to 5% by weight of lambda carrageenan, such as about 3% by weight of lambda carrageenan.

The film forming composition of the invention may comprise lambda carrageenan as only film forming polymer. In some embodiments, however, the film forming composition comprises a further film forming polymer, e.g. any of those described in US patent application No. 2007/0184093. For example, the film forming composition may comprise a further film forming polymer selected from e.g. alginate, modified cellulose, modified starch, pullulan, or iota carrageenan. In this case, it may be preferable to reduce somewhat the amount of lambda carrageenan.

A suitable alginate for inclusion in the film forming composition of the invention e.g. may be Protanal® LFR 5/60, which is a low viscosity sodium alginate. As an alternative, the alginate may be a higher viscosity alginate, such as Protanal® LF 10/60. Both alginates may be purchased from FMC BioPolymer Ltd.

For example, if e.g. 1% by weight of a second film former, such as iota carrageenan is included in the composition, the amount of lambda carrageenan may be reduced by about 1% by weight (based on the total weight of the film forming composition), provided that the film forming composition still comprises at least about 0.5% by weight of lambda carrageenan, or at least about 0.75% by weight of lambda carrageenan, preferably at least about 1% by weight of lambda carrageenan.

Thus, in some embodiments, the film forming composition comprises a second film forming polymer, such as iota carrageenan, at a concentration of generally less than 3% by weight, e.g. at a concentration of at most 2% by weight or at most 1% by weight. Preferably, in this case, the weight ratio between lambda carrageenan and the second film forming polymer(s), e.g. iota carrageenan, is 1:1 or higher, e.g. a weight ratio of 2:1 of lambda carrageenan: second film forming polymer(s).

The film forming composition of the invention also comprises a plasticizer with the general purpose of improving e.g. flexibility, solubility, water absorptiveness and wettability of the film product. The plasticizer preferably is present at a concentration of 1-15% by weight in the film forming composition. For example it may be present at a concentration of at least 1% by weight, at least 1.5% by weight, at least 2% by weight, at least 2.5% by weight or at least 3% by weight; and at most 15% by weight, at most 12% by weight, at most 10% by weight, at most 8% or at most 6% by weight; based on the total weight of the film forming composition before drying.

The weight ratio of plasticizer to film forming polymer (weight plasticizer: weight film forming polymer) e.g. may be from 10:1 to 1:5, or from 8:1 to 1:4, e.g. from 6:1 to 1:2, such as from 3:1 to 1:1.

The plasticizer may be selected e.g. from sugar alcohols and polyols, for example, glycerol, sorbitol, polyethylene glycol, propylene glycol and mixtures thereof. In some embodiments, the plasticizer is selected from sugar alcohols, e.g. sorbitol, and polyols, e.g. glycerol, polyethylene glycol, propylene glycol, as well as from mixtures of any of these.

The film formulation of the invention may comprise up to 85% by weight of the total dry film formulation, of one or several biologically active substances, e.g. up to 80% by weight, or up to 70% by weight, or up to 60% by weight, such as more than 5% by weight, more than 10% by weight, more than 20% by weight, more than 30% by weight, e.g. more than 40% by weight. It however should be understood, that it is also contemplated that the film formulation of the invention may contain a very low level of active ingredient, if this is for any reason desired, e.g. if the active ingredient is to be delivered at a very small dosage, e.g. from about 5% by weight to about 2% by weight, e.g. about 1% by weight, or about 0.01% by weight, or even lower.

By the term “biologically active substance”, as used herein, is meant any substance having a desired biological activity or effect when administered to a mammal subject in accordance with the invention. The substance may be e.g. a therapeutically active ingredient, or a diagnostic agent, e.g. an agent useful in an allergy test. It also may be a biologically active ingredient that nonetheless is not generally considered as a pharmaceutical, e.g. a naturopathic. As an example of a non-pharmaceutical active ingredient a stimulant or a nutraceutical may be mentioned, the latter generally defined as a substance that may be considered a food or part of a food and provides medical or health benefits, including the prevention and treatment of disease. Other biologically active substances may have both a therapeutical and a non-therapeutical use. For example, the biologically active substance for use according to the present invention may be e.g. a drug, a pharmaceutical, nutrition supplement, medication, vitamin, homeopathic remedy, herbal extract or remedy, and the like, or a mixture of any of these.

The biologically active substance may be a large or small, either water soluble or insoluble molecule. In case the biologically active substance is a pharmaceutically active ingredient, it may have a therapeutic activity making it suitable for the treatment of any of various diseases or health disorders. For example, it may be an antiphlogistic, analgesic, antipyretic, antibacterial, antiviral or anti-inflammation agent, e.g. a non-steroidal anti-inflammatory agent, or have an activity for the treatment of e.g. a CNS disorder, e.g. migraine, multiple sclerosis, Parkinson's disease, Alzheimer's diseases, depression, dementia, anxiety, insomnia, fatigue, etc. It also may be a therapeutic antibody for systemic action, transmucosal delivery of an antigen for vaccination or a hormone for hormonal therapy.

For example, the biologically active substance may be an antiphlogistic agent such as paracetamol, a non-steroidal anti-inflammatory agent, such as ibuprofen or diclofenac, or a sedative or hypnotic agent, such as zolpidem.

A herbal extract or remedy may be e.g. an extract from licorice, ginseng, menthol, ginkgo, cranberry, garlic, tobacco, coffee etc.

In one embodiment, the biologically active substance is one used in nicotine replacement therapy for smoking cessation, e.g. it is nicotine or a salt thereof, such as nicotine bitartrate (CAS Registry Number: 50915-69-0).

A nutritional supplement may be e.g. a water soluble vitamin, such as vitamin C, a lipid soluble vitamin, such as vitamin A, a mineral, such as selenium, an antioxidant, such as lycopene, etc.

It should be realized that the film formulation of the invention is by no means limited to the above-mentioned exemplary substances. Rather, it is contemplated that the film formulation of the invention will be suitable as a means of administration of any substance capable of transmucosal delivery, as will be recognized by the person of ordinary skill in the art. Indeed, by varying the amount of film forming polymer(s), plasticizers, fillers, taste masking agents and film thickness within the general limits defined herein, the film properties may be adjusted to produce a film that fulfills the criteria of a mucosal delivery film as described herein, irrespective of the chemical nature of the biologically active ingredient and the amount added per unit of film.

The film formulation of the present invention may comprise further ingredients, e.g. fillers, absorption enhancers, pH regulating agents, taste masking agents, solubilization agents, flavouring agents, sweetening agents and colouring agents. In case the film formulation is intended for pharmaceutical use any further ingredient must of course be pharmaceutically acceptable.

The total amount of additives incorporated into the film forming composition may be e.g. from 1% by weight up to 20% by weight, e.g. up to 10% by weight, or up to 5% by weight.

Suitable fillers (or bulking agents) are e.g. magnesium and calcium carbonate, calcium phosphate, calcium sulfate, clay, starch, chitosan and microcrystalline cellulose. The amount of filler incorporated into the film forming composition may be e.g. from 1% by weight up to 10% by weight, e.g. up to 5% by weight.

Preferably, the filler should have a small particle size, e.g. of about 100 μm or less, e.g. about 50 μm or less, in particular about 20 μm or less.

In one embodiment, the filler is microcrystalline cellulose, preferably microcrystalline cellulose having a particle size of about 20 μm or less.

In another embodiment, the filler is chitosan. Chitosan is a linear polysaccharide composed of randomly distributed β-(1-4)-linked D-glucosamine (deacetylated unit) and N-acetyl-D-glucosamine (acetylated unit). It is produced by N-deacetylation of the natural polymer chitin. The degree of deacetylation can be determined by NMR spectroscopy, and in commercial chitosans is in the range of 60 to 100%. The deacetylation process leads to a cationic polymer that is characterized by pH-dependent solubility in water; chitosan is only soluble in acidic aqueous solutions. Chitosan readily binds to mucosal membranes and may be used to enhance the transport of polar compounds across epithelial surfaces.

In some embodiments of the present invention chitosan is incorporated in the film formulation of the invention, e.g. to deliver biologically active substances to an acidic environment, such as in the vagina, where chitosan is dissolved and releases the biologically active substance.

The chitosan that is useful according to the invention e.g. may be 80-100% deacetylated, e.g. >90% deacetylated and have a particle size of, for example, 10-400 μm, e.g. 20-200 μm, such as about 40-100 μm.

The film formulation of the invention optionally may comprise a penetration (or absorption) enhancer to facilitate the absorption of the biologically active ingredient through the mucosal membrane. Examples of penetration enhancers that are considered useful according to the present invention include e.g. bile salts, surfactants, fatty acids and derivatives, chelators, cyclodextrins, chitosan and enzyme inhibitors.

Bile salts include sodium deoxycholate and sodium glycocholate (with or without EDTA); surfactants include sodium lauryl sulfate and sucrose laurate; fatty acids derivatives thereof include sodium laurate, sodium myristate, oleic acid and lauric acid and PEG derivatives thereof. Chelators include EDTA (ethylene diamine tetracetic acid), sodium salicylate, and sodium citrate. Cyclodextrins include α-, β- and γ-cyclodextrin, as well as hydroxypropyl cyclodextrin. Enzyme inhibitors include apotinin, bestatin, and bile salts.

A taste masking and penetration enhancing agent may be present in the film formulation of the invention. For example, the film formulation may comprise vitamin E polyethylene glycol-1000-succinate (d-alpha Tocopheryl Polyethylene Glycol 1000 Succinate; herein below referred to as TPGS) as a taste masking agent. TPGS is a waxy solid having a molecular weight of 1513 that may be prepared by esterification of the acid group of crystalline d-α-tocopheryl acid succinate by polyethylene glycol 1000. It has an aqueous solubility of 20% by weight and at a concentration in water of 0.02% by weight form normal micelles, while reversed micelles are formed at higher concentrations of TPGS.

In the present invention, TPGS may be used e.g. as an emulsifier, solubilizer, bio-availability enhancer and as a vehicle for a lipid-based drug formulation. It is a well-known component in pharmaceutical compositions, and may be used e.g. to improve drug bioavailability, as an emulsion vehicle, to reduce drug sensitivity on skin or tissues, as a solubilization agent, and as a taste masking agent. In some embodiments, TPGS is included in a film formulation of the invention containing a biologically active substance in order to improve the dispersion of said substance and its ability to form a homogeneous mixture together with the film former and plasticizer, if necessary, or to mask a bitter or otherwise disagreeable taste thereof. For example, TPGS may be included in the film forming composition at a concentration of 0.02 to 2% by weight, e.g. 0.05 to 1% by weight, or 0.1 to 0.5% by weight. When used to mask the taste of a substance or to improve its solubility, TPGS may be included e.g. in a 1:50 to 50:1 weight ratio to said substance, or a 1:10 to 10:1 weight ratio, or a 1:5 to 5:1 weight ratio, e.g. a 1:2 to 2:1 weight ratio.

Various agents capable of acting as vehicle or carriers for the biologically active substance, or as stabilizers thereof, may be used in the film-forming composition, e.g. insoluble substances capable of binding or encapsulating the biologically active substance, such as dextran microbeads, liposomes, or affinity binding agents, such as ion exchange agents, e.g. DEAE-cellulose. Proteins, e.g. albumin, may be added to stabilize sensitive substances, in particular proteins and polypeptides, such as insulin, growth hormones, blood clotting factors etc.

The film forming agent further may comprise a flavouring agent, a sweetening agent and/or a colouring agent, suitably of food quality and/or pharmaceutically acceptable. Examples of flavouring agents are peppermint, orange flavouring, and cherry flavouring. Examples of colouring agents are titanium dioxide and green, yellow or red food colour.

In some cases, the pH of the film forming composition may need to be adjusted, e.g. in case the biologically active agent to be included needs some specific pH in order to be stable or active. For example, the film forming composition may comprise a suitable pH buffering system, such as a phosphate buffer.

The above-mentioned examples of additives are not intended to form an exhaustive list of additives that may be incorporated in the film of the invention, and it is contemplated that other additives also may be added to the film forming composition of the invention, e.g. preservatives, surfactants, pH regulating agents. However, it is an advantageous feature of the invention that the film formulation has very satisfying shelf life even without addition of any preservatives and that surfactants are not necessary to obtain an excellent wettability and adhesiveness to moist biological surfaces such as the mucosal membrane.

The purified water to be used in the film forming composition e.g. may be distilled, demineralized or sterile-filtered water, e.g. pyrogen-free water of pharmaceutical quality.

As will be apparent to the person of ordinary skill in the art, the film forming composition as defined herein above may be prepared by admixing the components in various ways and orders, e.g. depending on the specific components to be included, and in the below, non-limiting Examples, several different modes of preparing the film forming compositions are shown. For example, lambda carrageenan and the plasticizer may be simply admixed in any order with water and kept under stirring conditions until lambda carrageen is fully hydrated, after which the biologically active substance and any further additive, such as a filler, a flavouring agent, a colouring agent etc., is added to the aqueous solution.

In case any further film forming component, e.g. iota carrageenan, is to be included, this may be added together with the lambda carrageenan.

Lambda carrageenan also may be dissolved separately in water and allowed time to become fully hydrated, while the active substance is admixed separately with the plasticizer, e.g. forming a dispersion therein, which dispersion is then admixed with the aqueous carrageenan solution.

So as to obtain a homogeneous film formulation, it is preferable that the lambda carrageenan has been completely dissolved and hydrated in the aqueous phase before adding biologically active substance and any other additives. For example, in order to obtain a fully dissolved and hydrated lambda carrageenan, a 2-3 hour stirring period at room temperature may be suitable.

The film forming composition of the invention may contain the biologically active substance in dissolved form, as a dispersion or as an emulsion, e.g. as an oil-in-water emulsion. Preferably, the biologically active substance is dissolved in the film forming composition, but may also be present in particulate form, e.g. as small particles, e.g. particles having a size of from 100 nm to 100 μm.

To prepare the film formulation of the invention, a layer of the film forming composition of the invention is distributed onto a solid surface, such as a glass plate, and allowed to dry. In general, the composition is applied to the surface to form an even, wet film having a thickness of about 1 mm to about 10 mm, in particular about 2 mm to about 5 mm.

After distributing the film forming composition onto the surface, the film layer is allowed to dry. At ambient temperature and normal atmospheric pressure, this may take about 10-30 hours, for example about 12-24 hours, e.g. about 16-20 hours. When dry, the film is easy to peel off from the surface using, for example, a sharp blade; the dry film does not break upon peeling off; and its surface is dry to the touch. The dry film generally has a film thickness of about 0.1 to about 1 mm, e.g. about 0.2 to about 0.5 mm.

The film formulation of the invention may be provided as a sheet or a strip, for example a strip which may be rolled and portioned using a suitable dispenser. The sheet or strip may be provided with e.g. grooves, dents or perforations so as to facilitate the separation of dosage units of suitable, predetermined size or may comprise printed size indications, e.g. grids or scares, to facilitate the cutting of suitable sizes. The film also may be provided with printed text matter or printed images, such as a brand name, a trade mark, a dosage indication, a symbol etc.

In some embodiments of the invention, the method for preparing a film formulation comprises dividing the film into separate dosage units, e.g. by cutting or punching.

The film dosage unit of the invention will have a suitable surface area, having regard to the concentration of the active ingredient within the film and the suitable dosage to be administered. As an example, a dosage unit having a surface area of from e.g. 1 cm² to 10 cm² may be selected, or from 4 cm² to 8 cm². It will be within the knowledge of the skilled person to adapt the size and shape of the film dosage unit having regard to such parameters as the loading of the biologically active substance within the film, fitting different mucosal sites and the required dosage.

The dosage provided by any particular film dosage unit will of course depend on various factors, e.g. the required dosage, the disease to be treated, the selected biological substance etc. In a dosage unit having a surface area of, say, 10 cm², it may be possible to include e.g. up to 1 g of biologically active substance. However, the maximum amount of course will vary between different substances and depend on factors such as film thickness and added formulation additives. As an example, however, a suitable film dosage unit of the invention may have a surface of about 6 cm² and contain e.g. 50-500 mg of a biologically active substance. Also, it should be realized that the film dosage unit may have any appropriate shape to match the site of administration of the active ingredient, e.g. it may be rectangular, circular, oblong, oval etc.

The dosage unit of the invention, when applied to the mucosal membrane in the oral cavity, will have a contact surface area that is generally equal to the surface area of the dosage unit. To reduce the required dosage unit area, one e.g. may increase the dose per unit area of mucosa and/or create a longer acting delivery product.

To reduce the contact surface area it also may be possible to fold the film of the invention one or several times or superposing several layers of the dry film at the time of preparing the film formulation or by the user before applying the film to its proper position on the mucosa. The dry film layers thus superposed may be simply adhered to each other by exerting a slight pressure. For example, film dosage units having a surface area of, say, 2 cm×5 cm and a thickness of e.g. 0.1 to 1 mm, containing a tobacco extract or nicotine, may be folded to be placed against the mucosal surface inside of the upper lip (oral vestibule), in a way that emulates normal use of snus.

It also is possible to superpose and press together films of the invention containing different biologically active substances, so as to obtain combination preparations. In this way, film formulations containing biologically active substances, e.g. substances requiring different, and perhaps mutually incompatible, conditions and additives during the film preparation may be prepared in a simple way. By physically separating biologically active substances according to this procedure unwanted interactions between substances during storage may also be avoided.

Thus, in some embodiments, the film formulation of the invention comprises several film layers, e.g. from 2 to 6 film layers, or from 2 to 4 layers, e.g. 3 layers, superposed and adhered to each other. Such multilayer film e.g. may have a thickness of about 0.2 to about 3 mm, e.g. about 0.3 to about 2 mm, or about 0.4 to about 1 mm, depending on the number of film layers and the thickness of each film layer.

Adherence of the layers to each other may be obtained by simply superposing the selected number of layers and applying a uniform, gentle pressure to the top of the sandwich structure thus obtained. Optionally, adherence between film layers may be further improved by humidification of the interface between successive film layers, e.g. by spraying water on the surface of a film layer before applying another film layer to it.

In a multilayer film of the invention, each film layer may be independently selected and the successive film layers may each be of a different composition. The multilayer film also may comprise one or more “empty” film layers, optionally containing excipients, such as colouring agents, taste-masking agents or buffering agents.

An “empty” film layer, interposed between two film layers comprising different biologically active agents, may serve the purpose of enhancing the separation of the active agents included in different layers, during storage of the film formulation and/or during the time of dissolution of the film formulation in contact with a moist mucosal membrane. In this way, consecutive delivery of more than one substance may be obtained. For example, in some embodiments, a multilayer film formulation is provided comprising one or more film layer sequence(s) wherein one first, fast dissolving film layer (e.g. to be facing the mucosal membrane) containing a first active ingredient is followed by a slower dissolving, empty film layer and then by a further film layer containing another active ingredient.

In some embodiments, a multilayer film formulation of the invention comprises one outer film layer of slower dissolution than the inner layer(s). By “outer film layer” is meant that on application of the film formulation of the invention to the mucosal membrane, the outer layer faces away from the mucosal membrane. In these embodiments the outer layer preferably will dissolve only after dissolution of the inner layers and thereby will minimize any loss of active substance to the surrounding lumen or cavity, e.g. to the saliva in the mouth, and additionally may help maintaining a high local concentration of the substance(s) released from inner film layer(s).

By a multilayer film as described herein above a multicompartment film dosage form is provided. In other embodiments, multicompartment film dosage forms are provided in essentially or wholly monolayer form. This may be achieved by superposing successive film layers over only a small portion, e.g. along the edges, creating films that are multicompartment essentially over the breadth rather than in height. In partly overlapping multilayer films of which the overlapping portion has a surface area that is much smaller than the surface area of the non-overlapping portion the dissolution profile will be essentially that of a monolayer film and yet components in the different portions of films may be kept separated from each other, in particular during storage. In such compartmentalized films, the separation of the active ingredients may be further improved by use of an intermediate, empty film layer, said empty film layer overlapping along its opposite edges with different films containing active ingredient.

Multicompartment films that are wholly monolayer also may be obtained by simply applying different film formulations side by side on a flat surface and allowing the films to flow into each other, or “fuse”, along the edges so as to obtain a “seamless” film on drying. In one embodiment, parallel bands of different film forming compositions are applied to a flat surface side by side in a manner that will allow the compositions to fuse along the edges so as to form a continuous band of on drying, which band may be transversally cut into suitably sized pieces. To enhance separation of active ingredients, any two bands containing different active ingredients may be separated by an “empty band” formed from a film forming composition containing no active ingredient, which band may be quite narrow.

In some embodiments, the “empty band” may be cast first and allowed to dry, and only then film forming compositions containing active ingredients are applied at each side of the empty band. The film forming compositions thus applied will flow against the edges of the empty band and on drying will form a continuous film comprising two parallel bands of different composition separated by an empty band.

The film formulation of the invention is suitable for transmucosal administration of numerous biologically active substances. It is a very advantageous feature that the inventive film, due to its flexibility, pliability and bioadhesivity, may be applied to the buccal, gingival and palate surface in the mouth. Moreover, once applied, the film of the invention very advantageously will remain in place even while the wearer is moving the mouth, e.g. speaking, for the whole period of film dissolution. In fact, due to the viscosity of the lambda carrageenan, even after dissolution is completed, the dissolved film formulation will remain in place for some further time, and this prolonged “residence time” which will further improve the transmucosal delivery. The active ingredient leaving the dissolving film formulation of the invention will diffuse across the mucosal membrane and reach the underlying tissue and the blood circulation, and thereby will allow for systemic administration while essentially avoiding first-pass metabolism and gastrointestinal digestion.

A surprising advantage of the inventive film formulation when used for the oral delivery of unpleasant, e.g. bitter, tasting substances is the substantially reduced taste perceived by the user. It is surmised that this is due to the tight adhesion of the film to the mucosal membrane and the prolonged residence time of the viscous film which will minimize contact of the unpleasant tasting substance with taste buds on the tongue.

### EXAMPLES

### Comparative Example 1

A composition not according to the invention was prepared using the following ingredients: 2 g kappa carrageenan with about 25% ester sulfate and about 34% 3,6-anhydrogalactose (3,6-AG),

3 g sorbitol

95 g distilled water

Kappa carrageenan and sorbitol were dissolved in distilled water and the solution was mixed thoroughly allowing the carrageenan to become fully hydrated. The wet mixture was applied to a horizontal glass plate at a thickness of 3 mm. The composition was air dried at room temperature for approximately 20 hours.

After 20 hours of drying, the kappa carrageenan formulation formed a rigid, water-containing gel. After an additional 24 hours drying period the gel dried into a hard crust. It was not possible to re-hydrate the dry crust at room temperature.

### Comparative Example 2

A composition not according to the invention was prepared using the following ingredients:

2 g iota carrageenan with an approximate 32% ester sulfate content and about 30% 3,6-AG

3 g sorbitol

95 g distilled water

The same procedure as in Comparative Example 1 was followed. The iota carrageenan formulation dried to a thixotropic gel with high water content. Further drying resulted in a thin, hard film that was not possible to re-hydrate at room temperature.

### Example 1

A film forming composition according to the invention, except for containing no biologically active substance, was prepared using the following ingredients:

2 g lambda carrageenan with an approximate 35% ester sulfate content and little or no 3,6-AG

3 g sorbitol

95 g distilled water

The same procedure as in Comparative Example 1 was followed. The lambda carrageenan formulation dried to a thin, flexible and clear film in about 20 hours. The thickness was about 0.1 mm, i.e. the film had dried into about 5% of the original thickness.

Surprisingly, the film adhered strongly to moist, oral tissue, where it was rapidly rehydrated and dissolved slowly. Adhered film had a smooth and pleasant mouth-feel and after about 5 minutes it was fully dissolved.

It is very surprising that lambda carrageenan, although based on the same, high molecular-weight linear galactose polymer structure as kappa and iota carrageenans, exhibits excellent film forming properties whereas the two other carrageenans do not.

### Example 2

Proceeding generally as in Example 1, films were prepared based on a number of film forming compositions containing various concentrations of lambda carrageenan. The wet film thickness was 2 mm.

The ingredients of each film forming composition, the thickness of the film produced, the time of dissolution of the film in contact with a moist oral mucosa and the mucosal adherence and other relevant physical properties are listed in Table 1.

TABLE 1

| Lambda | Sorbitol | Film | | |
|---|---|---|---|---|
| carrageenan | (% by | thickness | | Adherence and physical |
| (% by weight) | weight) | (mm) | Time to dissolve | properties |
| 7.5* | 3.0 | >0.1 | Did not dissolve | Very rigid. Did not adhere |
| | | | | well to wet mucosa |
| 5.0 | 3.0 | 0.15 | Dissolved in | Adheres well to wet mucosa. |
| | | | about 5 minutes | Easy to handle |
| 2.5 | 3.0 | 0.1 | Dissolved in | Adhered well to wet mucosa. |
| | | | about 5 minutes | Very flexible and |
| | | | | easy to handle. |
| 1.0 | 3.0 | <0.1 | Dissolved in less | Adhered well to wet mucosa. |
| | | | than 5 minutes | Some difficulties to |
| | | | | handle with ease. |
| 0.5* | 3.0 | <0.1 | NA | Too thin to enable easy |
| | | | | handling. |

*Not according to the invention.

This Example indicates that lambda carrageenan can be used to make film formulations having advantageous properties of easy handling and good adherence to wet mucosa. The Example is directed to a film formulation that contains only lambda carrageenan and sorbitol as a plasticizer and indicates that a suitable concentration interval of lambda carrageenan in film forming composition before drying is about 1 to 7% by weight, in particular about 2 to 6% by weight, or about 2.5 to about 5% by weight.

### Example 3

Proceeding generally as in Comparative Example 1, lambda carrageenan films according to the invention, except for containing no biologically active substance, were prepared containing different qualities of microcrystalline cellulose (from FMC Biopolymer Ltd) as filler. Each film forming composition contained 3.0 gram lambda carrageenan, 3.0 gram sorbitol and 3.0 gram of the microcrystalline cellulose specified in Table 2, in 41 gram distilled water. Table 2 also lists the mouth feel and properties of the films obtained.

TABLE 2

| Microcrystalline cellulose | | |
|---|---|---|
| (particle size, μm) | Film properties | Mouth Feel |
| Avicel ® PH-101 (50) | Hard and granular | Not pleasant. Poor adherence. |
| Avicel ® PH-102 (100) | Granular (“sand paper”) | Rough texture for oral mucosa |
| Avicel ® PH-105 (20) | Smooth surface | Very smooth and pleasant feel. |
| Avicel ® PH-301 (50) | Smooth film with rough | Rough texture for oral mucosa |
| | surface | |
| Avicel ® PH-302 (100) | Very rough surface | Not pleasant. Poor adherence. |
| Avicel ® PH-112 (100) | Very rough surface | Not pleasant. Poor adherence. |

All microcrystalline cellulose types were compatible with lambda carrageenan and resulted in strong and flexible films. Film formulations with Avicel® microcrystalline cellulose PH-105 showed surprisingly superior properties, compared to the other microcrystalline celluloses.

### Example 4

Proceeding generally as in Comparative Example 1, a film according to the invention, except for containing no biologically active substance, was prepared using chitosan (from Yantai Dongcheng Biochemicals Co., Ltd) as a filler.

The film forming composition contained the following ingredients:

3 g lambda carrageenan

5 g glycerol

5 g chitosan

87 g distilled water.

A solution of lambda carrageenan, glycerol and distilled water was prepared. Chitosan was suspended in the lambda carrageenan/glycerol solution and the mixture was applied to a glass plate at a thickness of 2 mm.

After drying for about 20 hours, a soft and strong film, approximately 0.25 mm thick, had formed.

The film adhered very well to oral mucosa, had a pleasant mouth feel and was fully dissolved after about 5 minutes in the mouth (placed against the buccal mucosa).

### Example 5

A film forming composition according to the invention was prepared using the following ingredients:

3 g lambda carrageenan

5 g glycerol

5 g paracetamol

87 g distilled water

Lambda carrageenan was dissolved in water and mixed thoroughly and allowed time to become fully hydrated. In a separate container, paracetamol was dispersed in glycerol. The paracetamol/glycerol dispersion was finally mixed with the aqueous carrageenan solution and applied at 3 mm thickness to a glass plate and allowed to air dry for about 24 hours. The total area corresponding to 100 ml wet film was approximately 333 cm².

After drying, the resulting film was about 0.4 mm thick, soft, flexible and easy to handle. The film adhered almost immediately and firmly to oral mucosa when placed inside the mouth and dissolved completely in about five minutes leaving no residues. In view of the very bitter taste of paracetamol, the taste perceived on dissolution of the film in the mouth was surprisingly faint. A film dose unit of 6 cm² contained about 90 mg of paracetamol.

### Example 6

A film forming composition according to the invention was prepared using the following ingredients:

3 g lambda carrageenan

5 g glycerol

6 g ibuprofen

87 g distilled water

The same procedure as in Example 5 was used to prepare the film formulation.

The dried film formulation was approximately 0.45 mm thick, strong, soft, flexible and easy to handle. It adhered almost immediately to oral mucosa (buccal placement) and remained in close contact with mucosa until dissolved completely in about five minutes. A film dose unit of 6 cm² contained about 110 mg of ibuprofen.

### Example 7

A film forming composition according to the invention was prepared using the following ingredients:

2.5 g lambda carrageenan

2.5 g glycerol

95 g distilled water

1 g diclofenac

1 g vitamin E polyethylene glycol-1000-succinate (TPGS)

Glycerol was dissolved in water. Lambda carrageenan was slowly added to the aqueous glycerol solution under constant stirring and was allowed time to swell in order to become fully dissolved. Diclofenac was mixed with pre-heated (approximately 45° C.) TPGS until diclofenac was fully dispersed. The dispersion was added to the preheated aqueous carrageenan/glycerol solution under constant mixing.

The film forming composition was applied to a glass plate at a thickness of 0.3 cm and air dried for about 20 hours.

The resulting film, having a thickness of approximately 0.2 mm, was strong, soft, flexible and easy to handle. It adhered almost immediately to the oral (buccal) mucosa and was fully dissolved in about five minutes, leaving no residues.

### Example 8

A film forming composition according to the invention was prepared using the following ingredients:

3 g lambda carrageenan

3 g sorbitol

3 g Avicel® microcrystalline cellulose, PH-105

91 g distilled water

Phosphate buffer, 0.1 M, pH 8.5

0.1 g nicotine bitartrate

Lambda carrageenan was slowly added to water under constant stirring, whereafter the sorbitol was added. The solution was left standing for 4 hours to allow the carrageenan to swell in order to become fully dissolved and hydrated. The microcrystalline cellulose was slowly added to the aqueous lambda carrageenan solution to produce a homogeneous dispersion of the cellulose.

Nicotine bitartrate was dissolved separately in a small volume of water and then added to the carrageenan/sorbitol/cellulose mixture under stirring.

The film forming composition was applied to a glass plate at a thickness of 0.2 cm and air dried for about 20 hours.

The film obtained was approximately 0.15 mm thick, flexible, soft and easy to handle. When applied to the buccal mucosa it adhered almost immediately and remained in close contact with the mucosa until fully dissolved, about 5 minutes after being placed into the mouth.

### Example 9

A film forming composition according to the invention was prepared using the following ingredients:

2.5 g lambda carrageenan

2.5 g glycerol

95 g distilled water

Approved food colour

Approved food flavor

1 g vitamin E polyethylene glycol-1000-succinate (TPGS)

1 g zolpidem

Lambda carrageenan was slowly added to the water under constant stirring and was allowed time to swell in order to become fully dissolved. Zolpidem was added to preheated TPGS (approximately 45° C.) until zolpidem was fully dispersed. The dispersion was added to the preheated water/glycerol solution under constant mixing. The food colour and flavor were finally added and the solution was thoroughly mixed.

The film forming composition was applied to a glass plate at a thickness of 0.2 cm and air dried for about 20 hours.

The film formulation obtained was strong, flexible, easy to handle and had a thickness of approximately 0.2 mm. It adhered almost immediately when applied to the buccal mucosa of the mouth and remained in close contact with mucosa until fully dissolved, about five minutes after being placed into the mouth.

### Example 10

A film forming composition according to the invention was prepared using the following ingredients:

1 g lambda carrageenan

1 g iota carrageenan

2 g glycerol

96 g distilled water

6 g paracetamol

Lambda carrageenan and iota carrageenan were slowly added to water under constant stirring and were allowed time to swell in order to become fully dissolved. Paracetamol was dispersed in glycerol and added to the lambda carrageenan/iota carrageenan water solution under constant mixing.

The film forming composition was applied to a glass plate at a thickness of 0.3 cm and air dried for about 20 hours. The thickness of the dry film was approximately 0.3 mm. The film formulation was soft and flexible, adhered rapidly and strongly to buccal mucosa and dissolved completely in about five minutes.

### Example 11

A film forming composition according to the invention was prepared using the following ingredients:

3 g lambda carrageenan

3 g glycerol

0.5 g lyophilized coffee

95 g distilled water

Essentially the same procedure as in Example 8 was used. Lambda carrageenan and glycerol were fully dissolved in water. Lyophilized coffee was then added and the solution was stirred.

The film forming composition was applied to a glass plate at a thickness of 0.2 cm and air dried for about 20 hours.

The thickness of the dried film formulation was approximately 0.15 mm.

The film formulation was soft, flexible, easy to handle and adhered almost immediately to oral (buccal) mucosa. The film remained tightly associated with the oral mucosa until fully dissolved about five minutes after application.

### Example 12

A film forming composition according to the invention, except for containing no biologically active substance, was prepared using the following ingredients:

3 g lambda carrageenan

5 g glycerol

5 g Avicel® PH-105

87 g distilled water

The film formulation was prepared essentially as in Example 8.

The film was analyzed directly after drying and after six months storage at room temperature.

The flexibility, tensile strength, mouth feel, easiness to handle, oral mucosa adherence and overall appearance was almost identical before and after storage. A slight increase in dissolution time (about 15%) was observed.

Not only was the film formulation very stable at room temperature, but bacterial and fungal contamination of the formulation was completely absent after six months storage. This is in strong contrast to the wet, gel formulations that typically deteriorate with microbial growth after about one week under the same storage conditions. Fungal growth is characterized by a visible mold (typically yellow, black or blue molds) growing inside the wet film and/or on the surface of the wet film. Bacterial growth is characterized by wet films becoming opaque and producing a “milky” solution when dissolved in purified water.

### Example 13

A film formulation according to the invention was prepared using the following ingredients:

3 g lambda carrageenan

3 g glycerol

10 sachets of Kronan Snus, from Swedish Match

85 g distilled water

The sachets of snus (a moist tobacco powder) were extracted with water at room temperature. Lambda carrageenan and glycerol were added to the water soluble snus extract and stirred until fully dissolved.

The film forming composition was applied to a glass plate at a thickness of 0.2 cm and air dried for about 20 hours.

The obtained film formulation was approximately 0.2 mm thick, dark brown, almost without smell, soft, flexible, easy to handle, strong and has a good mouth feel. When placed into the mouth (in contact with, for example, buccal mucosa) the formulation adhered rapidly and remained in close contact with mucosa until completely dissolved after about five minutes.

### Example 14

A film forming composition according to the invention was prepared, comprising:

3 g lambda carrageenan

5 g glycerol

5 g paracetamol

87 g distilled water

The composition was processed essentially as in Example 5, casting the wet mixture at a thickness of 0.5 cm.

A dose unit of 6 cm² contained approximately 167 mg of paracetamol. The dried film thickness was about 0.5 mm. When placed into the mouth (buccal positioning) the film was completely dissolved after about 10 minutes.

### Example 15

A film forming composition according to the invention was prepared, comprising

2 g lambda carrageenan

10 g glycerol

5 g paracetamol

83 g distilled water

The wet mixture was applied to a horizontal glass plate at a thickness of 0.2 cm. The dried film (about 0.25 mm thick) was folded three times under mild compression to reduce the dose unit area from 18 cm² to 6 cm², giving approximately 333 mg paracetamol per 6 cm² dose unit. The folded film was approximately 0.75 mm thick, soft, flexible, easy to handle and adhered well to buccal mucosa. The folded film was completely dissolved after about 15 minutes of contact with buccal mucosa.

### Example 16

A film forming composition according to the invention was prepared, comprising

2 g lambda carrageenan

25 g ibuprofen

5 g glycerol

68 g purified water

Lambda carrageenan was slowly added to water under constant stirring. The solution was allowed 4 hours to swell under occasional stirring to allow the carrageenan to become fully swelled and hydrated.

Ibuprofen was added to glycerol and mixed until a uniform dispersion was obtained. The ibuprofen/glycerol dispersion was mixed into the carrageenan lambda solution until a homogeneous dispersion was formed.

The film forming composition was applied to a glass plate at a thickness of 0.2 cm and air dried for about 20 hours.

The film obtained was approximately 0.6 mm thick, flexible, soft and easy to handle. When applied to the buccal mucosa it adhered almost immediately and remained in close contact with the mucosa until fully dissolved, about 10 minutes after being placed into the mouth.

In the dry film, about 80% by weight is the biologically active ingredient ibuprofen.

### Example 17

A film forming composition according to the invention was prepared, comprising

2 g lambda carrageenan

15 g nicotine bitartrate

5 g glycerol

68 g purified water

Lambda carrageenan was slowly added to water under constant stirring. The solution was allowed 4 hours to swell under occasional stirring to allow the carrageenan to become fully swelled and hydrated.

Nicotine bitartrate was added to glycerol and mixed until a uniform dispersion was obtained. The nicotine bitartrate/glycerol dispersion was mixed into the carrageenan lambda solution until a homogeneous dispersion was formed.

The film forming composition was applied to a glass plate at a thickness of 0.2 cm and air dried for about 20 hours.

The film obtained was approximately 0.6 mm thick, flexible, soft and easy to handle. When applied to the buccal mucosa it adhered almost immediately and remained in close contact with the mucosa until fully dissolved, about 7 minutes after being placed into the mouth.

In the dry film, about 70% by weight is the biologically active ingredient nicotine bitartrate.

### Example 18

A film forming composition according to the invention, except for containing no biologically active substance, was prepared using the following ingredients:

3 g lambda carrageenan

3 g glycerol

94 g distilled water

Lambda carrageenan and glycerol were dissolved in distilled water and the solution was mixed thoroughly, allowing the carrageenan to become fully hydrated. One 20-ml portion of the solution was mixed with green food colour; another 20-ml portion was mixed with red food colour. The two portions and a third portion, to which no colorant was added, were applied to three horizontal glass plates, each film forming composition at a thickness of 2 mm. The wet films on the three glass plates were air dried at room temperature for approximately 24 hours, giving three dry films, each having a thickness of about 0.1 mm. The films were soft, flexible and strong, and pieces thereof adhered swiftly to the mucosa when placed in the mouth and dissolved in about 7 minutes in contact with the mucosa.

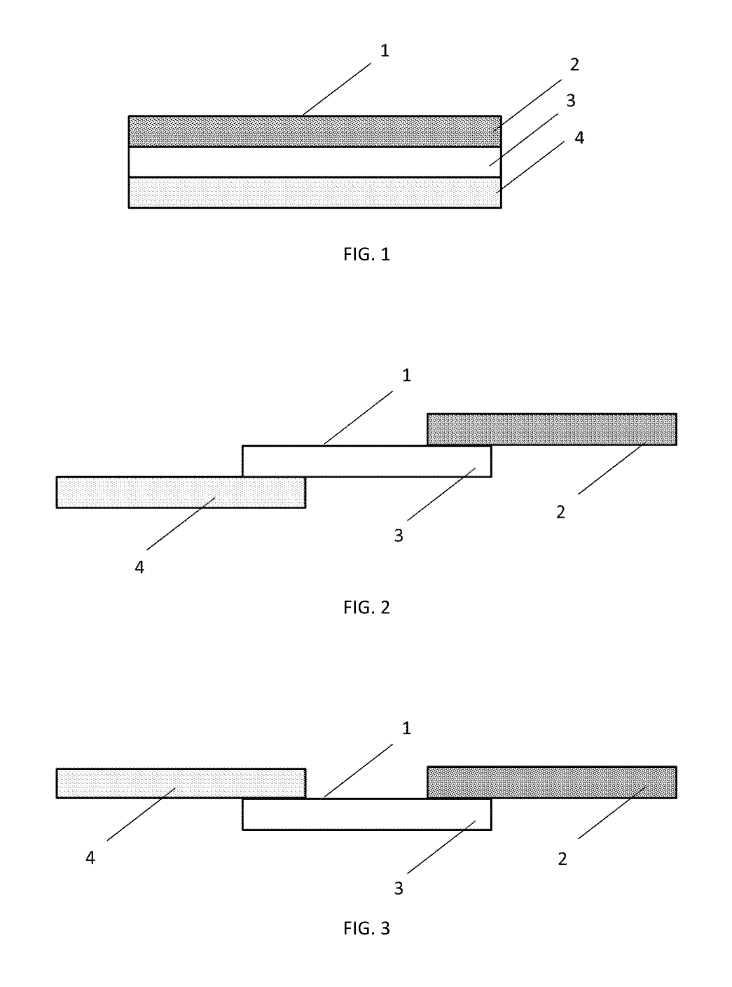

A multilayer film preparation 1 as illustrated in FIG. 1 was prepared using the three dry films.

On a flat surface, e.g. a glass plate, the three dry films, one red, one green and one uncoloured, were superposed in a sandwich fashion, with the uncoloured film 3 in the middle, flanked by the green film 2 and the red film 4, respectively, applying only a very gentle pressure thereto. Very surprisingly, when superposed the three film layers 2, 3, 4 adhered to each other without necessity for applying any moisture or elevated pressure. Pieces of suitable form and size could then be cut from the obtained sandwich structure film 1. Moreover, the obtained multilayer film 1 could be folded, rolled and generally manhandled without the layers 2, 3, 4 separating from each other or being dislodged. In the mouth, a piece of the sandwich film 1 adhered to the mucosa and dissolved within about 15 minutes.

In this example, the two colorants represent different active ingredients and the example shows that sandwich films containing more than one ingredient may easily be prepared according to the invention. The uncoloured layer 3 in between the red and the green film layers 4, 5 allow for the active ingredients being kept separated from each other during storage of the film.

### Example 19

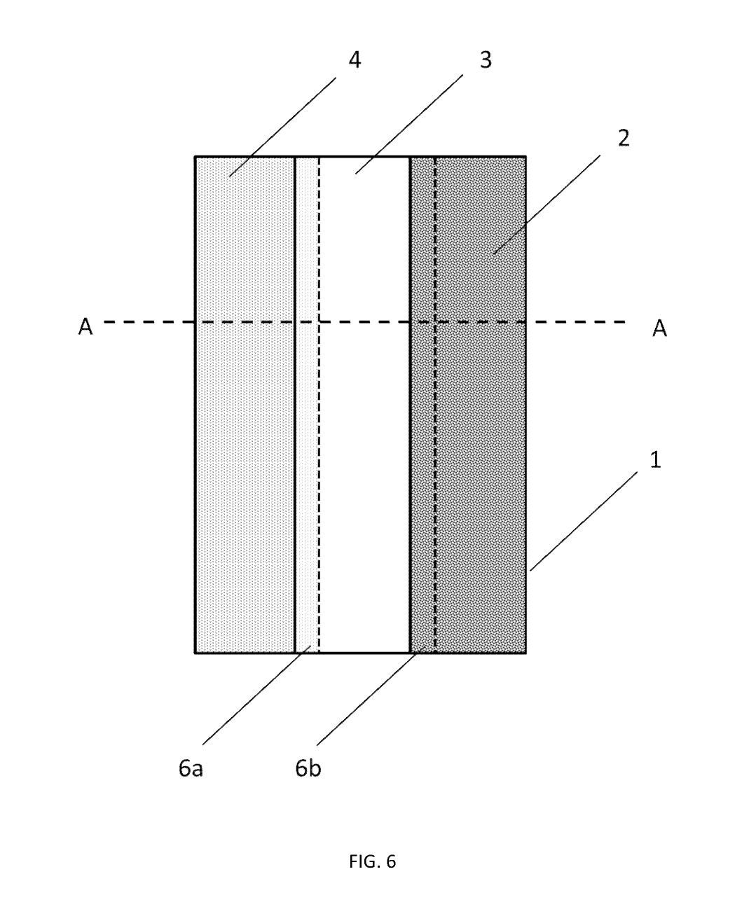

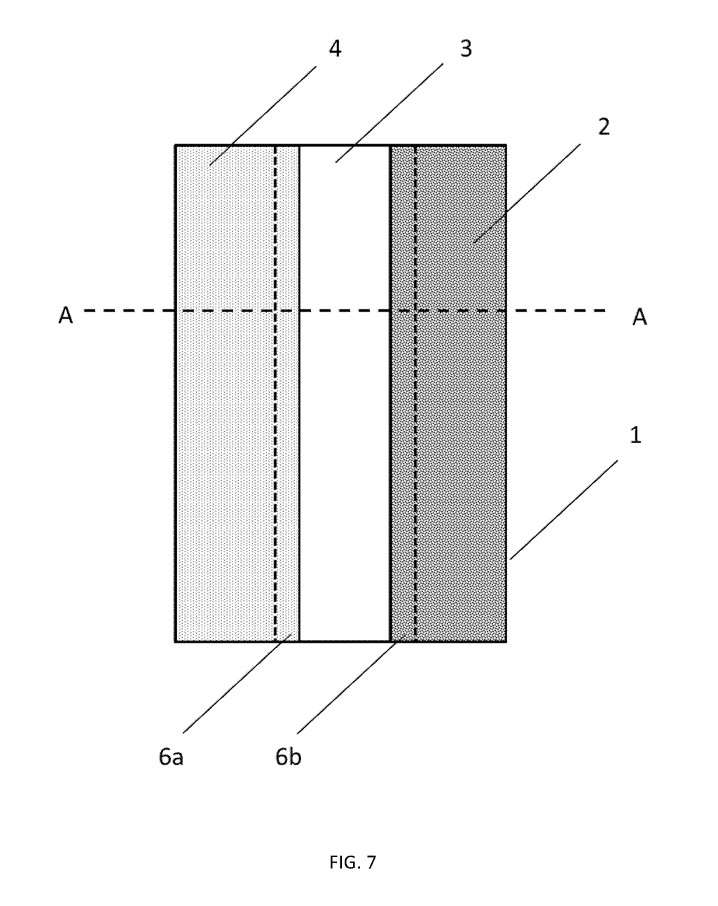

Two multilayer films according to Example 19 are illustrated in FIGS. 2, 3, 6 and 7, respectively. The only difference between the multilayer film of FIGS. 2 and 6, on the one hand, and FIGS. 3 and 7, on the other, is the way the film layers 2, 3 and 4 have been superimposed.

Pieces of the three monolayer films 2, 3, 4 of Example 18 (one green, one red, one uncoloured) were cut and superposed in a partly overlapping fashion, to obtain a multilayer film preparation 1 whereby the region of overlap constituted only narrow bands 6a, 6b at the edges. The pieces were arranged so that the uncoloured film 3 overlapped along one edge with the green film 2 and along the opposite edge with the red film 4. In the region of overlap, the films were made to adhere to each other by applying only a very gentle pressure. The resulting multilayer film 1 had essentially the features of a monolayer film, except for in the narrow regions of overlap 6a, 6b which were of bilayer structure. The film structure supported handling without the film layers 2, 3, 4 being dislodged or falling apart. When a piece of the multilayer film 1 was introduced into the mouth it adhered to the oral mucosa and dissolved within about 7 minutes, i.e. in about the same time as one of the individual films 2, 3 or 4.

### Example 20

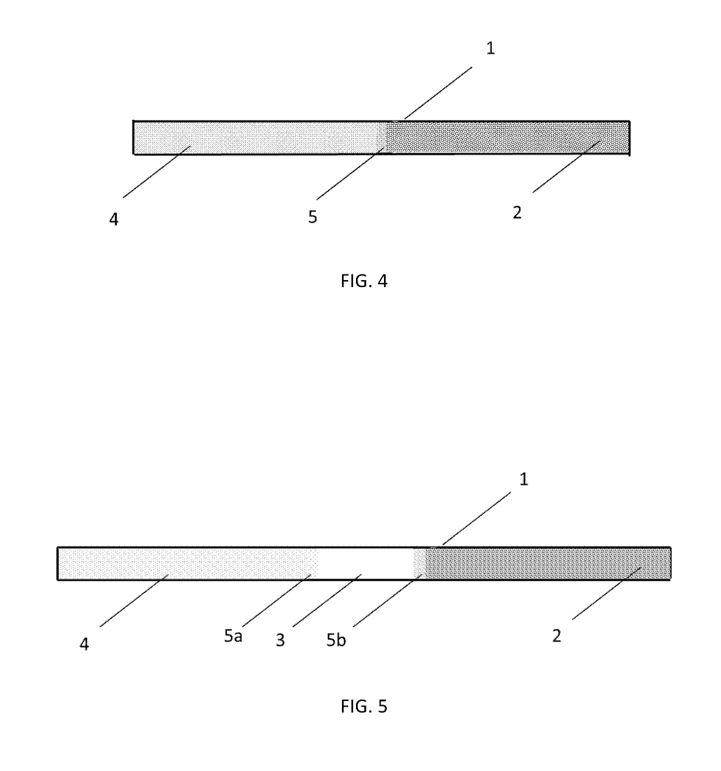

A monolayer film as prepared in Example 20 is illustrated in FIG. 4.

Two differently coloured film forming compositions were prepared as in Example 18 and were poured onto a casting glass plate, at each side of a separating plate arranged orthogonally against the casting glass plate (the two glass plates forming together an inverted “T”). After pouring the two film forming compositions onto the casting glass plate, the separating plate was carefully removed. The still wet film forming compositions flew into each other at the edges only, forming a continuous monolayer film 1 on drying, where the green coloured area 2 and the red couloured area 4 were adjacent to each other, with a narrow band 5 of transition from one colour to the other.

### Example 21

A monolayer film as prepared in Example 21 is illustrated in FIG. 5.

Example 20 is repeated but using instead two separating plates parallel to each other and at a small distance from each other. In the area between the two separating plates, uncoloured film forming composition is poured, while green and red film forming composition, respectively, is poured on the opposite sides of the two separating plates. After pouring the three film forming compositions onto the casting glass plate, the separating plates are carefully removed. The still wet film forming compositions flow into each other at the edges only, forming a continuous monolayer film 1 on drying, having a green coloured area 2 and a red couloured area 4 separated by an uncoloured area 3, with narrow bands of transition 5a, 5b on each side.

### Example 22

A narrow, elongated strip of an uncoloured film according to the invention is placed on a glass plate, and two differently coloured film forming compositions, prepared as in Example 18, are poured onto the plate, one colour at each side of the strip. The film forming compositions flow into and adhere to the strip along its two edges, forming a continuous film on drying, having a green coloured area and a red couloured area separated by an uncoloured area.

The above examples of multilayer films or multiarea films should be seen as exemplary only and to the person of ordinary skill in the art several other combinations and variations will present themselves. For example, multilayer films comprising more than three different layers may be prepared, or films comprising layers of different thickness or different dissolution profiles, or films comprising portions or areas having different numbers of layers. Likewise, the film portions or areas or film layers may independently comprise any number of active ingredients or other substances, e.g. taste masking agents or flavouring agents. Furthermore, combinations of different, partly overlapping sandwich structures may be prepared. Thus, what should transpire is the great flexibility, adaptability and multitude of choices offered by the film preparations of the invention. Indeed, by the multilayer and/or multiarea films, formulations may be provided wherein one and the same dosage unit may comprise several substances that preferably should be kept apart during storage but may—or should—come into contact with each other on administration. The multicompartment (multilayer or multiarea) films of the invention also provide for the possibility of including, in one and the same dosage unit, compounds that require different, and perhaps mutually incompatible, conditions for optimal shelf life, e.g. compounds that require an acidic pH in one compartment and compounds that require a basic pH in another. Furthermore, as seen in Example 18, the time of dissolution of the film may be adjusted by increasing the number of layers in a sandwich structure film. Additionally, the delivery of different substances in a given order may be obtained by appropriately selecting the order in which the film layers are arranged in a multilayer structure.

In one embodiment, thus, a multilayer film is provided having one layer, here termed the “contact layer”, the outer surface of which is to be brought into contact with the mucosa when administered to a mammal subject, and another layer, here termed the “outer layer”, at the opposite side of the multilayer film, i.e. facing away from the mucosa to which the film is adhered, the outer surface of which has been treated to render it essentially non-adherent. Thereby, the directed application of the film is ensured and inadvertent mistakes may be avoided. For example, the outer surface of the outer layer may have been treated to reduce its capability to take up water and adhere, by, for example, applying thereto a thin covering of vegetable fat. Alternatively, the outer layer may comprise an additive, e.g. a filler, that provides decreased adherence.

In still another embodiment, a multilayer film is provided having one layer, facing away from the mucosa, that provides a slower dissolution, e.g. by containing a higher concentration of film forming agent in the film, such as from 6 to 12% by weight of lambda carrageenan, covering a layer of higher dissolution rate. In this fashion a multilayer film may be obtained having an outer layer that dissolves only after dissolution of the inner film layer. The outer layer may or may not comprise an active ingredient.

It may be added that while the above examples show that the dry films of the invention may be adhered to each other by applying only a gentle pressure to them, adherence may be further improved by humidifying the surface of one or both film layers before bringing them into contact with each other, e.g. by spraying of distillated water.

1. A method of preparing a mucoadhesive film formulation by
preparing a film forming composition comprising
(i) lambda carrageenan as a film forming polymer, at a concentration of about 1-7% by weight,
(ii) a plasticizer at a concentration of about 1-15% by weight,
(iii) a biologically active substance, and
(iv) purified water;
distributing the film forming composition as a wet film layer onto a solid surface; and
allowing the film layer to dry.

2. The method according to claim 1, wherein lambda carrageenan is present at a concentration of 2-6% by weight.

3. The method according to claim 1, wherein the plasticizer is selected from sugar alcohols, polyols, polyethylene glycol, propylene glycol, or is a mixture of any of these.

4. The method according to claim 1, wherein the film forming composition comprises a filler.

5. The method according to claim 4, wherein the filler is selected from magnesium and calcium carbonate, calcium phosphate, calcium sulfate, clay, starch, chitosan and microcrystalline cellulose

6. The method according to claim 5, wherein the filler is microcrystalline cellulose having a particle size of about 20 μm.

7. The method according to claim 1, wherein the film forming composition comprises a flavouring agent, a colouring agent, a pH regulating agent, a penetration enhancer, a solubilization agent, a sweetening agent, and/or a taste-masking agent.

8. The method according to claim 1, wherein the biologically active substance is a pharmaceutical substance, a nutraceutical substance, nutrition supplement, medication, vitamin, homeopathic remedy, herbal extract or remedy, and the like, or a mixture of any of these

9. The method according to claim 8, wherein the biologically active substance is a pharmaceutical substance.

10. The method according to claim 1, wherein the biologically active substance is present in the film forming composition in an amount so as to provide a concentration of biologically active substance in mucoadhesive film formulation of 0.001 to 85% by weight.

11. The method according to claim 1, wherein the film forming composition comprises a film forming polymer selected from alginate, modified cellulose, modified starch, pullulan, and iota carrageenan or a mixture of any of these.

12. The method according to claim 1, comprising superposing two film layers over at least a portion of their surface and allowing the film layers to adhere to each other in the surface of overlap, so as to obtain a multilayer or partially multilayer film.

13. The method according to claim 1, comprising dividing the film into separate or separable dosage units.

14. A mucoadhesive film formulation obtainable by a method according to claim 1.

15. A mucoadhesive film dosage unit obtainable by the method according to claim 13.

16. The method according to claim 2, wherein the plasticizer is selected from sugar alcohols, polyols, polyethylene glycol, propylene glycol, or is a mixture of any of these.

17. The method according to claim 2, wherein the film forming composition comprises a filler.

18. The method according to claim 2, wherein the film forming composition comprises a flavouring agent, a colouring agent, a pH regulating agent, a penetration enhancer, a solubilization agent, a sweetening agent, and/or a taste-masking agent.
